(19) United States
(12) Patent Application Publication
(10) Pub. No.: US 2026/0117374 A1
(43) Pub. Date: Apr. 30, 2026

(54) METHOD FOR RECYCLING AN EFFLUENT GAS FROM CHEMICAL VAPOR DEPOSITION OR INFILTRATION

(71) Applicant: SAFRAN LANDING SYSTEMS, VELIZY-VILLACOUBLAY (FR)

(72) Inventors: Laurent MAISSE, MOISSY-CRAMAYEL (FR); Paul-André CHEVRIN, MOISSY-CRAMAYEL (FR); Inès BARON, LYON (FR); Jean-François Daniel René POTIN, MOISSY-CRAMAYEL (FR)

(21) Appl. No.: 19/474,360

(22) PCT Filed: Apr. 9, 2024
(86) PCT No.: PCT/FR2024/050467

§ 371 Date: Oct. 10, 2025

(30) Foreign Application Priority Data
Apr. 11, 2023 (FR) FR2303565

(51) Int. Cl. C23C 16/44; C07C 5/02; C23C 16/04; C23C 16/26

(52) U.S. Cl. CPC C23C 16/4412; C07C 5/02; C23C 16/045; C23C 16/26

(57) ABSTRACT

A method for recycling an effluent gas from chemical vapor deposition or infiltration of pyrocarbon and including dihydrogen, C₁ to C₄ light unsaturated hydrocarbons and C₅ and higher heavy hydrocarbons, the method including a) removing heavy hydrocarbons from the effluent gas, b) catalytically hydrogenating the light unsaturated hydrocarbons so as to remove them by transforming them into C₁ to C₄ light saturated hydrocarbons, this catalytic hydrogenation being carried out on the effluent gas obtained after carrying out procedure a) and consuming some of the dihydrogen present in the effluent gas, and c) adding the effluent gas obtained after carrying out procedure b) to a reaction chamber and forming pyrocarbon by chemical vapor deposition or infiltration using the C₁ to C₄ light saturated hydrocarbons present in the effluent gas thus added.

### TECHNICAL FIELD

The invention relates to chemical vapor deposition or infiltration techniques (“Chemical Vapor Deposition” or “CVD” and “Chemical Vapor Infiltration” or “CVI”) for the formation of pyrolytic carbon. More specifically, the invention relates to the recycling of the effluent gas from this technique with a view to obtaining, from this recycled gas, high-quality pyrolytic carbon. The invention finds particular application in the manufacture of carbon/carbon composite material parts, in particular friction parts such as brake discs for aircraft or automobiles.

### PRIOR ART

The technique of manufacturing carbon/carbon by pyrocarbon densification uses as raw material reagents from the light hydrocarbon family such as natural gas, propane, butane, or more generally alkanes, chosen in order to optimize the chemical vapor infiltration reaction, in particular its material yield, and the kinetics of the reaction. The reagents are added into a reaction chamber where densification takes place. After their passage and the deposition reaction in the reaction chamber, the effluent gas is extracted from the installation.

The method for densification by pyrocarbon by chemical vapor infiltration uses conditions that crack the reactive gas molecules in the reaction chamber, thus releasing the carbon atoms and promoting their deposition on the fibers. The material yield of the method is low: approximately 8% of the carbon atoms in the incoming gases are deposited. The remaining carbon atoms recombine into numerous hydrocarbon compounds and leave the reaction chamber in the form of an effluent gas. The infiltration time required to obtain a carbon/carbon material can be several hundred hours.

The effluent gas contains a high proportion of methane and dihydrogen, but also a large number of hydrocarbons, including unsaturated (alkenes, alkynes), aromatics and heavy hydrocarbons (C₅ and higher), in low concentration. The complexity of this mixture, some of whose compounds are harmful, and others tend to condense when cooling in the installations in the form of oils or solids, leads in most cases to destroying them by combustion in the oxygen of the air in order to recover the energy in thermal form.

The principle of recycling the effluent gas to reinject it as a reactant into the reaction chamber has been proposed in the literature. In this regard, mention can be made of U.S. Pat. No. 8,084,079 which proposes reintroduction into the gas phase, admitted at the inlet of the reaction chamber, of at least a fraction of the gas flow extracted from the effluent gas. Before its reintroduction, the heavy hydrocarbons are removed from the effluent gas, by oil washing or condensation. Nevertheless, it is possible to improve the quality of the material produced by this recycling as well as the densification kinetics.

### DISCLOSURE OF THE INVENTION

The present invention relates to a method for recycling an effluent gas resulting from chemical vapor deposition or infiltration of pyrocarbon and comprising dihydrogen, C₁ to C₄ light unsaturated hydrocarbons and C₅ and higher heavy hydrocarbons, comprising the following steps:
a) removing the heavy hydrocarbons from the effluent gas,
b) catalytically hydrogenating the light unsaturated hydrocarbons so as to remove them by transforming them into C₁ to C₄ light saturated hydrocarbons, this catalytic hydrogenation step being carried out on the effluent gas obtained after carrying out step a) and consuming some of the dihydrogen present in said effluent gas, and
c) adding the effluent gas obtained after carrying out step b) to a reaction chamber and forming pyrocarbon by chemical vapor deposition or infiltration using the C₁ to C₄ light saturated hydrocarbons present in the effluent gas thus added.

The invention proposes to introduce into the reaction chamber a recycled effluent gas having a composition closer to that of the initial gas than in the solution of patent U.S. Pat. No. 8,084,079. The invention is remarkable in particular in that it proposes, after the removal of heavy hydrocarbons, a catalytic hydrogenation to remove the light unsaturated hydrocarbons and transform them into light saturated hydrocarbons useful for deposition. The consumption of unsaturates during catalytic hydrogenation allows to improve the quality of the material produced from the recycled gas and the consumption of dihydrogen, which is possibly supplemented by a subsequent removal of all or some of the unconsumed dihydrogen, produces an increase in the deposition kinetics. This step takes advantage of the presence of dihydrogen in the effluent gas to chemically transform, without the input of external material or energy, the unsaturated hydrocarbons into hydrocarbons beneficial for deposition. Recycling according to the invention allows to considerably reduce the consumption, and therefore the price, of reactive gases without degrading the quality of the material produced or the kinetics of the reaction. The invention also contributes to a reduction in the production of hydrocarbon residues intended for combustion, thus reducing the amount of greenhouse gases emitted into the atmosphere.

In an exemplary embodiment, compression of the effluent gas obtained after carrying out step a) is carried out before implementation of step b) to bring it to a pressure of at least 4 bar, for example at least 6 bar or even comprised between 4 bar and 12 bar or between 6 bar and 12 bar.

Such a characteristic advantageously allows to promote catalytic hydrogenation and to bring the composition of the treated effluent gas even closer to that of the initial gas.

In an exemplary embodiment, removal of residual dihydrogen from the effluent gas is carried out after carrying out step b) and before step c).

Such a feature allows to further increase the deposition kinetics of pyrocarbon formed from the recycled gas.

In an exemplary embodiment, densification of a fiber preform into carbon fibers by pyrocarbon is carried out during step c).

The present invention also relates to a chemical vapor deposition or infiltration installation, comprising:
a first reaction chamber in which pyrocarbon is intended to be formed by chemical vapor deposition or infiltration having an inlet through which a reaction gas phase is intended to be admitted and an outlet through which an effluent gas is intended to be evacuated, and
a circuit for treating and recycling the effluent gas defining a loop between the outlet of the first reaction chamber and an inlet of a second reaction chamber which corresponds to the first reaction chamber or to a chamber separate therefrom, said circuit for treating and recycling the effluent gas comprising (i) a device for removing C₅ and higher heavy hydrocarbons from the effluent gas having an inlet in communication with the outlet of the first reaction chamber and an outlet, and (ii) a catalytic hydrogenation device having an inlet in communication with the outlet of the device for removing heavy hydrocarbons and an outlet in communication with the inlet of the second reaction chamber.

This installation may be intended for the implementation of a method as described above.

In an exemplary embodiment, the circuit for treating and recycling the effluent gas further comprises a compressor located between the device for removing C₅ and higher heavy hydrocarbons and the catalytic hydrogenation device.

In an exemplary embodiment, the circuit for treating and recycling the effluent gas further comprises a device for removing residual dihydrogen located between the catalytic hydrogenation device and the inlet of the second reaction chamber.

### BRIEF DESCRIPTION OF THE DRAWINGS

FIG. 1 schematically and partially illustrates steps of an example of a method according to the invention.

### DESCRIPTION OF THE EMBODIMENTS

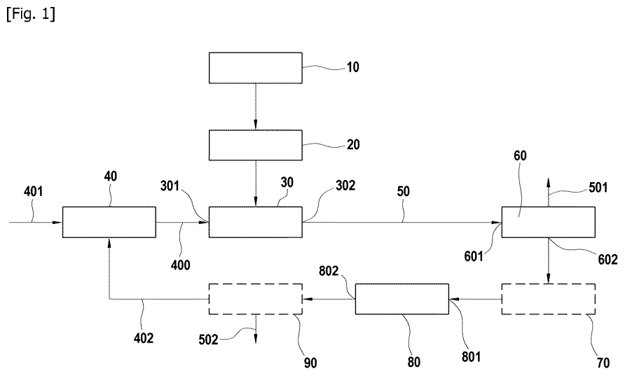

An example of a method and installation according to the invention will be described in connection with FIG. 1. This example relates to the densification of porous carbon fiber substrates by pyrocarbon so as to obtain parts made of carbon/carbon composite material, but the person skilled in the art will recognize that the invention is not limited to this case, also covering the chemical vapor deposition of pyrocarbon on the surface of substrates to be coated.

Fibers 10 of carbon or of a carbon precursor undergo one or more textile operations, for example in a loom 20, so as to obtain a fiber preform having the shape of the part to be obtained and intended to form the fiber reinforcement thereof. The preform is produced by textile operations known per se depending on the intended application. For example, a plurality of annular fiber layers can be superimposed or a helical texture can be wound in flat turns to form superimposed annular layers and then a connection can be carried out by needling. Other techniques can be implemented such as the formation of the fiber preform by three-dimensional weaving. “Three-dimensional weaving” or “3D weaving” means a weaving method by which at least some of the warp yarns bind weft yarns over several weft layers. A reversal of roles between warp and weft is possible in this text and must be considered as covered within the scope of the invention.

When the textile operation(s) are carried out on carbon precursor fibers, the latter are transformed into carbon fibers by carbonization heat treatment at a temperature typically comprised between 750° C. and 1100° C.

The fiber preform(s) to be densified are positioned in a reaction chamber 30 of a chemical vapor infiltration furnace to carry out their densification. Densification is carried out by introducing a reaction gas phase 400 through an inlet 301 of the reaction chamber 30. The gas phase 400 is obtained at the outlet of a gas mixer 40 receiving on a first inlet a gas 401 of nominal composition and on a second inlet a recycled effluent gas 402. At the start of the densification, the gas phase 400 is only constituted by the gas 401 and, as it progresses, effluent gas is recycled and this recycled gas 402 forms in whole or in part the gas phase 400 added into the chamber 30. A method is thus proposed in which the recycling of the effluent gas is carried out continuously during a cycle of formation of pyrocarbon in the vapor phase so that the effluent gas thus recycled is used during this cycle. The gas 401 may typically be a mixture of methane and propane. A temperature comprised approximately between 850° C. and 1050° C. and a reduced pressure comprised approximately between 0.5 kPa and 3.3 kPa may be imposed in the chamber 30. The formation of pyrocarbon in the chamber 30 is accompanied by the generation of by-products, such as dihydrogen from the decomposition of the pyrocarbon precursor gas, C₁ to C₄ light unsaturated hydrocarbons and heavy hydrocarbons having at least 5 carbon atoms, such as polycyclic aromatic hydrocarbons (“PAHs”), benzene hydrocarbons or tars. An effluent gas 50 containing these by-products as well as unreacted C₁ to C₄ light saturated hydrocarbons is discharged through an outlet 302 of the chamber 30. By way of illustration, the composition of the effluent gas 50 at the outlet 302 of the chamber 30, expressed in volume percentages, may be as follows:
light saturated hydrocarbons in an amount of 50% to 70%,
light unsaturated hydrocarbons in an amount of 5% to 10%,
heavy hydrocarbons in an amount of less than 2%, and
dihydrogen in an amount of 20% to 40%.

The installation here comprises a circuit for treating and recycling the effluent gas 50 defining a loop between the outlet 302 and the inlet 301 of the chamber 30 which allows to obtain the recycled effluent gas 402 which is reintroduced into the chamber 30 (via the gas mixer 40) to be used to form additional pyrocarbon. The following focuses on providing details relating to the recycling implemented in the illustrated installation example.

First of all, the heavy hydrocarbons 501 are removed from the effluent gas 50 (step a)). This removal corresponds to a technique known per se. The effluent gas 50 passes through a suitable device 60 which has an inlet 601 in communication with the outlet 302 of the reaction chamber 30. The removal can be carried out by at least one of washing the gas 50 with a capture liquid, such as an oil, or by condensation. As an example, reference may be made to patent U.S. Pat. No. 8,084,079 which describes a possible solution for removing the heavy hydrocarbons from the effluent gas 50.

In the example illustrated, the installation comprises a compressor 70 in communication with an outlet 602 of the device 60 for removing heavy hydrocarbons 501. The effluent gas 50 obtained after carrying out step a) passes through the compressor 70 to be brought to a pressure of at least 4 bar, for example at least 6 bar or at least 10 bar. This pressure may be comprised between 4 bar and 12 bar, for example between 6 bar and 12 bar or between 10 bar and 12 bar. As indicated above, this compression allows to promote catalytic hydrogenation but is not, however, obligatory.

The installation comprises a catalytic hydrogenation device 80 comprising an inlet 801 in communication with the outlet 602 (and with the compressor 70 in the illustrated example). The effluent gas 50, obtained after carrying out step a) and after a possible passage through the compressor 70, undergoes catalytic hydrogenation (step b)). In the illustrated example, the gas 50 is in the compressed state at the pressure values indicated above during the catalytic hydrogenation. Catalytic hydrogenation takes advantage of the presence of hydrogen in the gas 50 to remove light unsaturated hydrocarbons (alkenes and alkynes) by transforming them into light alkanes, such as ethane, propane or butane. This reaction requires neither the input of external material, since hydrogen is present in sufficient amount in the mixture, nor the input of energy, since the reaction is exothermic.

Partial hydrogenations of unsaturated hydrocarbons are known reactions in petroleum refining operations and in the production of major intermediates for petrochemicals. Indeed, it is desired to remove the most unsaturated hydrocarbons (alkynes) from light olefinic petroleum fractions to enable their use in petrochemicals or in the polymer industry, where very high olefin purities are required. In these techniques, the main goal is to remove triple bonds, and not to push the hydrogenation too far, in order to conserve ethylene, which is then used for polymer synthesis.

The catalytic hydrogenation method proposed here seeks a more complete hydrogenation of unsaturates compared to those operations known to transform them into saturated hydrocarbons. Such a more complete hydrogenation can be obtained:
by increasing the residence time in the reactor,
by increasing the total specific surface area of catalyst,
by increasing the temperature up to a certain limit.

For example, palladium or a palladium-based catalyst may be used as a catalyst, although other catalysts may also be considered.

Generally speaking, the effluent gas obtained after implementing step b) may advantageously have a volume content of light unsaturated hydrocarbons less than or equal to 10%, for example less than or equal to 8%.

In the example illustrated, the residual dihydrogen 502 is removed from the effluent gas obtained after implementing step b) using a suitable device 90. The person skilled in the art will recognize that several methods are possible for carrying out this removal. For example, the dihydrogen can be separated from the rest of the effluent gas by membrane separation, which allows for a very high-quality separation. Other techniques can be used, such as cryogenic distillation or cryogenic condensation, to carry out this separation. It will be noted that the removal of the residual dihydrogen is not mandatory since the dihydrogen has a slowing effect on the pyrocarbon deposition kinetics but does not affect the quality and properties of the deposition. The presence of residual dihydrogen in the effluent gas added into the furnace can thus be accepted if the implementation of a longer densification time can be considered. Moreover, it will be noted that the gas 401 can advantageously be devoid of dihydrogen or incorporate a small amount thereof, which allows to reduce the proportion of dihydrogen in the gas phase 400 resulting from the mixture between the gas 401 and the recycled gas 402. Generally speaking, supplementing the recycled gas 402 with the gas 401 allows to compensate for the flow rate of gas lost during the treatment and maintain the total flow rate entering the reaction chamber. It will be noted that the gas 401 can be in the minority by having a mass content lower than that of the gas 402 in the reaction mixture at the inlet of the chamber. The mass content of the gas 401, in this reaction mixture, can be less than or equal to 30%, for example less than or equal to 20% or 10%. The use of gas 401 is even optional and the system can operate in a closed loop, in the latter case it may be sought to remove the residual dihydrogen in the effluent gas after carrying out step b).

In the case where compression of the effluent gas has been carried out beforehand, expansion of the treated effluent gas can be carried out, for example after the possible removal of residual dihydrogen, before implementation of step c) of introduction into the chamber 30. Following this expansion, the pressure of the effluent gas can be increased to at most 4 bar, for example to at most 3.5 bar, for example to a pressure comprised between 3 bar and 4 bar or between 3 bar and 3.5 bar. The recycled gas 402 is added through the inlet 301 of the chamber 30 which is in communication with the outlet 802 of the catalytic hydrogenation device 80 (and with the device for separating the residual dihydrogen 90 in the example considered).

By way of illustration, the composition of the recycled effluent gas 402 which is intended to be reintroduced into the chamber 30, expressed in volume percentages, may be as follows:
light saturated hydrocarbons in an amount of 72% to 83% in the case where there is no removal of residual dihydrogen after catalytic hydrogenation, or in an amount of 95% to 99% when such removal is carried out,
residual light unsaturated hydrocarbons, possibly still present, in an amount of at most 1%,
residual heavy hydrocarbons, possibly still present, in an amount of at most 1%,
dihydrogen in an amount of not more than 28% in the case where there is no removal of residual dihydrogen after catalytic hydrogenation, or in an amount of not more than 2% when such removal is carried out.

Generally speaking, the recycling which is the object of the invention produces a significant reduction in the amount of light unsaturated hydrocarbons and heavy hydrocarbons, between the outlet 302 and the inlet 301 of the chamber 30, by providing a recycled effluent gas 402 having a volume content of at most 1% in light unsaturated hydrocarbons and at most 1% in heavy hydrocarbons.

The invention is remarkable in that it proposes a single loop intended to treat the effluent gas from the chamber 30 to transform it into a gas of composition close to the gas 401 of nominal composition without external input of material, and minimal input of energy limited to the possible compression of the treated gas upstream of the catalytic hydrogenation.

The formed part can be made of a carbon-carbon composite material. For example, it could be a friction part such as an aircraft or automobile brake disc, a nozzle throat, or a heat shield.

An example has been described in which the treated and recycled effluent gas is reintroduced into the same reaction chamber, but it does not go beyond the scope of the invention if it is added into a reaction chamber separate from the one from which it originates. In this case, the installation comprises a first reaction chamber similar to the chamber 30 described above and at least one second reaction chamber, separate from the first reaction chamber, into which the treated and recycled effluent gas is added. Thus, in this case, the effluent gas obtained after implementing step b) is added into the second reaction chamber and pyrocarbon is formed by chemical vapor deposition or infiltration using the C₁ to C₄ light saturated hydrocarbons present in the effluent gas thus added into the second reaction chamber. In this case, the installation comprises several reaction chambers and the treatment and recycling circuit is shared between the different chambers. Generally speaking, the effluent gas from different chambers can be treated and reused in any one or more chambers of the installation.

The expression “comprised between . . . and . . . ” must be understood as including the limits.

1. A method for recycling an effluent gas from chemical vapor deposition or infiltration of pyrocarbon and comprising dihydrogen, C₁ to C₄ light unsaturated hydrocarbons and C₅ and higher heavy hydrocarbons, comprising:
a) removing the heavy hydrocarbons from the effluent gas,
b) catalytically hydrogenating the light unsaturated hydrocarbons so as to remove them by transforming them into C₁ to C₄ light saturated hydrocarbons, said catalytic hydrogenation step being carried out on the effluent gas obtained after carrying out step a) and consuming some of the dihydrogen present in said effluent gas, and
c) adding the effluent gas obtained after carrying out step b) to a reaction chamber and forming pyrocarbon by chemical vapor deposition or infiltration using the C₁ to C₄ light saturated hydrocarbons present in the effluent gas thus added.

2. The method according to claim 1, wherein a compression of the effluent gas obtained after carrying out step a) is carried out before implementation of step b) to bring it to a pressure of at least 4 bar.

3. The method according to claim 1, wherein a removal of the residual dihydrogen from the effluent gas is carried out after carrying out step b) and before step c).

4. The method according to claim 1, wherein a densification of a fiber preform into carbon fibers by pyrocarbon is carried out during step c).

5. A chemical vapor deposition or infiltration installation, comprising:
a first reaction chamber in which pyrocarbon is intended to be formed by chemical vapor deposition or infiltration having an inlet through which a reaction gas phase is intended to be admitted and an outlet through which an effluent gas is intended to be evacuated, and
a circuit for treating and recycling the effluent gas defining a loop between the outlet of the first reaction chamber and an inlet of a second reaction chamber which corresponds to the first reaction chamber or to a chamber separate therefrom, said circuit for treating and recycling the effluent gas comprising (i) a device for removing C₅ and higher heavy hydrocarbons from the effluent gas having an inlet in communication with the outlet of the first reaction chamber and an outlet, and (ii) a catalytic hydrogenation device having an inlet in communication with the outlet of the device for removing heavy hydrocarbons and an outlet in communication with the inlet of the second reaction chamber.

6. The installation according to claim 5, wherein the circuit for treating and recycling the effluent gas further comprises a compressor located between the device for removing C₅ and higher heavy hydrocarbons and the catalytic hydrogenation device.

7. The installation according to claim 5, wherein the circuit for treating and recycling the effluent gas further comprises a device for removing residual dihydrogen located between the catalytic hydrogenation device and the inlet of the second reaction chamber.
